(19) United States
(12) Patent Application Publication
(10) Pub. No.: US 2024/0325854 A1
(43) Pub. Date: Oct. 3, 2024

(54) METHOD OF ASSESSING UPPER EXTREMITY EXERCISE ABILITY OF USER THROUGH REHABILITATION CONTENT AND COMPUTING APPARATUS FOR PERFORMING THE METHOD

(71) Applicant: Electronics and Telecommunications Research Institute, Daejeon (KR)

(72) Inventors: Hang Kee KIM, Daejeon (KR); Dongchun Lee, Daejeon (KR); Ki Suk Lee, Daejeon (KR)

(73) Assignee: Electronics and Telecommunications Research Institute, Daejeon (KR)

(21) Appl. No.: 18/431,586

(22) Filed: Feb. 2, 2024

(30) Foreign Application Priority Data
Mar. 30, 2023 (KR) 10-2023-0041803

(51) Int. Cl. A63B 71/06; A63B 24/00; G06F 3/01; G06T 11/00

(52) U.S. Cl. CPC A63B 71/0622; A63B 24/0062; G06F 3/011; G06T 11/00; A63B 2071/0638; A63B 2071/0666; A63B 2220/803

(57) ABSTRACT

Disclosed is a method of assessing an upper extremity that assesses an upper extremity exercise ability of a user by providing rehabilitation content to the user who is wearing a virtual reality (VR)/augmented reality (AR)-based visual device. The method provides the rehabilitation content including four types of exercise assessment methods according to various rehabilitation scenarios through the VR/AR-based visual device the user wears and simultaneously assesses the upper extremity exercise ability of the user and a rehabilitation therapy state by recognizing a position and a shape of a hand of the user moving while the user is wearing the VR/AR-based visual device according to the provided rehabilitation content.

### CROSS-REFERENCE TO RELATED APPLICATIONS

This application claims the benefit of Korean Patent Application No. 10-2023-0041803 filed on Mar. 30, 2023, in the Korean Intellectual Property Office, the entire disclosure of which is incorporated herein by reference for all purposes.

### BACKGROUND

### 1. Field of the Invention

One or more embodiments relate to a method of assessing an upper extremity, and more particularly, to technology for assessing an upper extremity exercise ability of a user through rehabilitation content executed by a virtual reality (VR)/augmented reality (AR)-based visual device.

### 2. Description of the Related Art

A stroke patient undergoes a long-term rehabilitation process after initial surgery. There are various methods of rehabilitation, but among the methods, a rehabilitation method using virtual reality (VR)/augmented reality (AR) technology is commonly used. The rehabilitation method using VR/AR technology may perform an assessment in various ways to identify the condition of a patient by performing the rehabilitation process on the patient and measuring the rehabilitation effect on the patient.

Specifically, the Fugl-Meyer assessment (FMA) scale is used to assess the exercise function of the upper extremity and lower extremity of a stroke patient. The Berg balance scale (BBS) is used to measure the balance ability of the lower extremity. The modified Barthel index (MBI) assesses a patient's ability to perform motion in daily life.

However, as the scale tool used in this rehabilitation process performs a rehabilitation therapy and condition measurement separately, the burden on a patient increases. That is, the FMA scale, which is widely used to assess an upper extremity exercise ability, takes more than 20 minutes to check 33 items, which is a relatively large number of items, and becomes less useful due to the accumulation of patient fatigue. Recently, to solve this problem, a method of reducing items to 37 items or 12 items has been introduced, and a method of using machine learning to reduce and assess 10 items including an upper extremity and lower extremity is also being studied. However, all these methods still burden a patient in that these methods perform a rehabilitation therapy and assessment separately.

### SUMMARY

Embodiments provide a method of assessing an upper extremity exercise ability while performing rehabilitation exercises through rehabilitation content executed by a virtual reality (VR)/augmented reality (AR)-based visual device.

According to an aspect, there is provided a method of assessing an upper extremity, the method including determining rehabilitation content to assess an upper extremity exercise ability of a user in a pre-set rehabilitation scenario, determining a virtual scenario item to interact with the user through the determined rehabilitation content, providing the virtual scenario item through a virtual reality (VR)/augmented reality (AR)-based visual device the user wears by adjusting a virtual position of the virtual scenario item based on body information of the user, recognizing movement of the user according to the virtual scenario item provided through the VR/AR-based visual device, and assessing the upper extremity exercise ability of the user by analyzing the movement of the user.

The determining of the virtual scenario item may include determining one virtual scenario item among a virtual object touchable by the user, a virtual image panel, and an adjustment apparatus wearable by the user to assess the upper extremity exercise ability of the user according to a body part of the user.

The providing of the virtual scenario item may include, when the virtual scenario item is a virtual image panel, adjusting a position and a direction of the virtual image panel based on the body information of the user and spatial information where the user is positioned and providing the virtual image panel of which the position and the direction are adjusted to the user through the VR/AR-based visual device.

The providing of the virtual scenario item may include, when the virtual scenario item is a virtual object, setting a touchable area by a hand of the user based on the body information of the user and providing the virtual object displayable on the touchable area to the user through the VR/AR-based visual device.

The providing of the virtual scenario item may include, when the virtual scenario item is an adjustment apparatus, setting a starting position and an ending position of the adjustment apparatus based on a posture of the user and providing the starting position and the ending position of the adjustment apparatus to the user through the VR/AR-based visual device to assess a moving path of the adjustment apparatus held by a hand of the user.

The recognizing of the movement of the user may include recognizing movement of the user as to whether the virtual image panel is touched by a palm of the user in response to the position and the direction of the virtual image panel that is visualized through the VR/AR-based visual device.

The recognizing of the movement of the user may include recognizing movement of the user as to whether the virtual object is touched by a finger or hand of the user in response to the virtual object that is visualized through the VR/AR-based visual device.

The recognizing of the movement of the user may include recognizing movement of the user as to whether the adjustment apparatus held by the hand of the user is moved according to the starting position and the ending position of the adjustment apparatus.

The assessing of the upper extremity exercise ability of the user may include deriving a score on the upper extremity exercise ability of the user by analyzing movement of the user corresponding to the rehabilitation content based on the pre-set rehabilitation scenario and assessing the upper extremity exercise ability of the user in response to the derived score.

According to another aspect, there is provided a computing apparatus for performing a method of assessing an upper extremity, the computing apparatus including a processor, in which the processor is configured to determine rehabilitation content to assess an upper extremity exercise ability of a user in a pre-set rehabilitation scenario, determine a virtual scenario item to interact with the user through the determined rehabilitation content, provide the virtual scenario item through a VR/AR-based visual device the user wears by adjusting a virtual position of the virtual scenario item based on body information of the user, recognize movement of the user according to the virtual scenario item provided through the VR/AR-based visual device, and assess the upper extremity exercise ability of the user by analyzing the movement of the user.

The processor may be configured to determine one virtual scenario item among a virtual object touchable by the user, a virtual image panel, and an adjustment apparatus wearable by the user to assess the upper extremity exercise ability of the user according to a body part of the user.

The processor may be configured to, when the virtual scenario item is a virtual image panel, adjust a position and a direction of the virtual image panel based on the body information of the user and spatial information where the user is positioned and provide the virtual image panel of which the position and the direction are adjusted to the user through the VR/AR-based visual device.

The processor may be configured to, when the virtual scenario item is a virtual object, set a touchable area by a hand of the user based on the body information of the user and provide the virtual object displayable on the touchable area to the user through the VR/AR-based visual device.

The processor may be configured to, when the virtual scenario item is an adjustment apparatus, set a starting position and an ending position of the adjustment apparatus based on a posture of the user and provide the starting position and the ending position of the adjustment apparatus to the user through the VR/AR-based visual device to assess a moving path of the adjustment apparatus held by a hand of the user.

The processor may be configured to recognize movement of the user as to whether the virtual image panel is touched by a palm of the user in response to the position and the direction of the virtual image panel that is visualized through the VR/AR-based visual device.

The processor may be configured to recognize movement of the user as to whether the virtual object is touched by a finger or hand of the user in response to the virtual object that is visualized through the VR/AR-based visual device.

The processor may be configured to recognize movement of the user as to whether the adjustment apparatus held by the hand of the user is moved according to the starting position and the ending position of the adjustment apparatus.

The processor may be configured to derive a score on the upper extremity exercise ability of the user by analyzing movement of the user corresponding to the rehabilitation content based on the pre-set rehabilitation scenario and assess the upper extremity exercise ability of the user in response to the derived score.

According to still another aspect, there is provided a system for assessing an upper extremity, the system including a VR/AR-based visual device configured to play immersive rehabilitation content in a space where a user is positioned and a computing apparatus configured to assess an upper extremity exercise ability of the user by recognizing movement of the user through the VR/AR-based visual device, in which the computing apparatus is configured to provide rehabilitation content through the VR/AR-based visual device the user wears based on body information of the user and assess the upper extremity exercise ability of the user by analyzing movement of the user according to the rehabilitation content provided through the VR/AR-based visual device.

Additional aspects of embodiments will be set forth in part in the description which follows and, in part, will be apparent from the description, or may be learned by practice of the disclosure.

According to embodiments, since an upper extremity exercise ability of a user is assessed by performing rehabilitation exercises through rehabilitation content executed by a virtual reality (VR)/augmented reality (AR)-based visual device, a rehabilitation assessment and ability assessment of the user may be performed simultaneously.

According to embodiments, since an upper extremity exercise ability of a user is assessed like playing a game through immersive rehabilitation content, a smooth assessment may be performed by increasing fun and interest while the user's the recognition of rehabilitation exercise.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating a system for assessing an upper extremity of a user through rehabilitation content executed by a virtual reality (VR)/augmented reality (AR)-based visual device, according to an embodiment;

FIG. 2 is a diagram illustrating detailed operations of a computing apparatus to assess an upper extremity exercise ability of a user, according to an embodiment;

FIG. 3 is a diagram illustrating a method of providing a touchable object through upper extremity exercise of a user using rehabilitation content, according to an embodiment;

FIG. 4 is a diagram illustrating a method of providing a touchable object through upper extremity exercise of a user using rehabilitation content, according to another embodiment;

FIG. 5 is a diagram illustrating a method of providing a touchable object through upper extremity exercise of a user using rehabilitation content, according to another embodiment;

FIG. 6 is a diagram illustrating a method of inducing and recognizing the hand touch of a user using a virtual image panel, according to an embodiment; and

FIG. 7 is a flowchart illustrating a method of assessing an upper extremity, according to an embodiment.

### DETAILED DESCRIPTION

Hereinafter, embodiments will be described in detail with reference to the accompanying drawings.

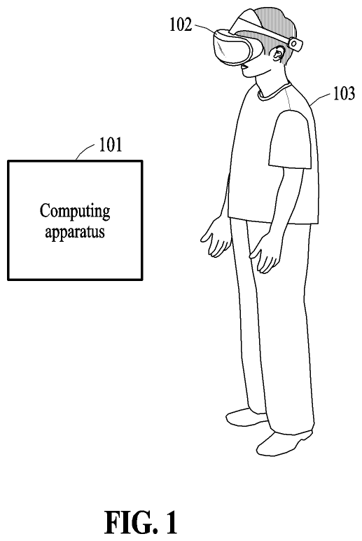

FIG. 1 is a diagram illustrating a system for assessing an upper extremity of a user through rehabilitation content executed by a virtual reality (VR)/augmented reality (AR)-based visual device, according to an embodiment.

Referring to FIG. 1, a computing apparatus 101 may determine rehabilitation content to assess an upper extremity exercise ability of a user in a pre-set rehabilitation scenario. The computing apparatus 101 may determine a virtual scenario item to interact with the user through the rehabilitation content. The computing apparatus 101 may provide the virtual scenario item through a VR/AR-based visual device the user wears by adjusting a virtual position of the virtual scenario item based on body information of the user. The VR/AR-based visual device may be a device capable of recognizing the position and shape of the hand of the user while the user is wearing the VR/AR-based visual device. For example, the VR/AR-based visual device is a head-mounted display (HMD) and may be Meta Quest 2, Meta Quest Pro, HoloLens, HoloLens 2, etc.

The computing apparatus 101 may analyze the position and angle of each body part and joint of the user. More specifically, when assessing the upper extremity exercise ability of the user, the computing apparatus 101 may verify body numerical information of the user to measure the posture of the user accurately. Here, the body numerical information of the user may include the shoulder width, arm length, forearm length, upper arm length, waist length, leg length, thigh length, shin length, etc. For example, the computing apparatus 101 may verify the body numerical information of the user measured in advance or measure the body numerical information of the user in real time through a joint recognition sensor such as Kinect, etc.

The computing apparatus 101 may recognize the movement of the user according to the virtual scenario item provided through the VR/AR-based visual device. The computing apparatus 101 may be implemented in four types to assess the upper extremity exercise ability of the user. The four types may be as follows.

The first type may verify the basic posture to assess the upper extremity exercise ability of the user by analyzing and recognizing the position and/or angle of each body part and joint of the user.

The second type may verify whether an image panel is touched by the palm of the user after adjusting the position and direction of the image panel that is implemented in the palm shape and capable of being displayed in the virtual space.

The third type may verify whether an object is touched by the finger or hand of the user after adjusting the position and direction of the object touchable in the virtual space.

The fourth type may display a moving position in the virtual space for an adjustment apparatus (a controller and/or smartphone) and verify that the adjustment apparatus moves along the moving position.

The computing apparatus 101 may assess the upper extremity exercise ability of the user by analyzing the movement of the user. The computing apparatus 101 may simultaneously assess the upper extremity exercise ability of the user and the rehabilitation therapy state by recognizing the position and shape of the hand of the user moving while the user is wearing the VR/AR-based visual device according to the rehabilitation content.

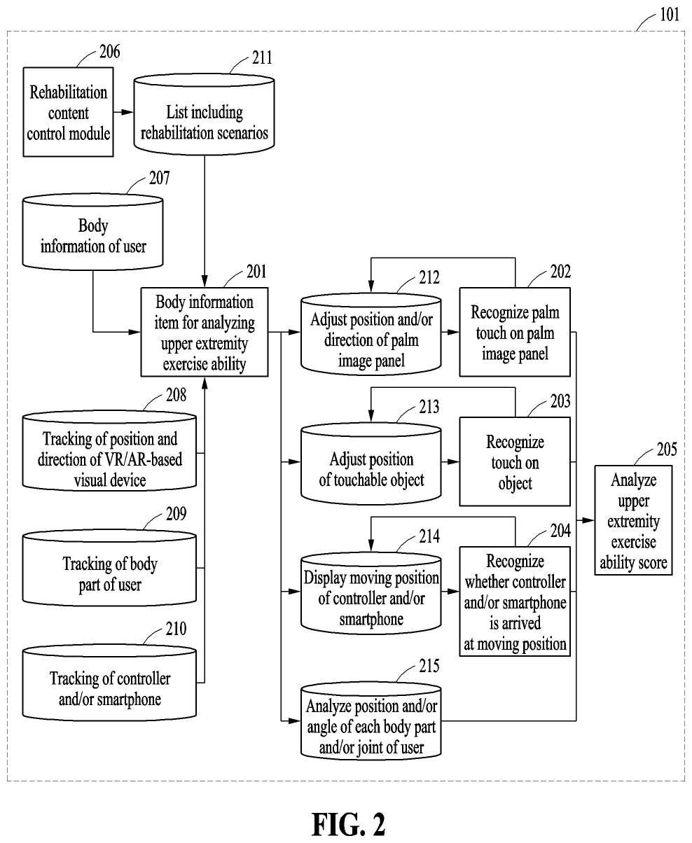

FIG. 2 is a diagram illustrating detailed operations of a computing apparatus to assess the upper extremity exercise ability of the user, according to an embodiment.

In operation 201, the computing apparatus 101 may select a scenario item to analyze the upper extremity exercise ability of the user. The computing apparatus 101 may determine rehabilitation content using a list including a plurality of rehabilitation scenarios 211, body information of a user 207, a tracking of a position and direction of a VR/AR-based visual device 208, a tracking of a body part of a user 209, and a tracking of an adjustment apparatus 210. The list including the plurality of rehabilitation scenarios 211 may be interoperated with a rehabilitation content control module 206, and the rehabilitation content control module 206 may include rehabilitation content such as popping a balloon by moving the hand, wrist stability, etc.

The computing apparatus 101 may select one piece of rehabilitation content from various rehabilitation scenarios according to the list including the plurality of rehabilitation scenarios 211 and select a scenario item corresponding to the selected piece of rehabilitation content. Here, the scenario item may be an item purely made for a therapeutic purpose or a scenario item that may assess the upper extremity exercise ability of the user proposed herein. Accordingly, when the scenario item is selected to assess the upper extremity exercise ability of the user, the computing apparatus 101 may analyze the assessment score on the upper extremity exercise ability of the user in addition to the therapy of the user. The computing apparatus 101 may assess the upper extremity exercise ability of the user by selecting one of four types of analysis methods depending on the purpose.

### 1) First Type

In operation 215, the computing apparatus 101 may identify the postures of the user by analyzing the position and angle of each body part and joint of the user and determine a posture corresponding to an assessment among the identified postures of the user. The computing apparatus 101 may determine the posture score on the upper extremity exercise ability of the user based on the determined posture.

For example, in the case of shoulder flexion to a 90 degrees (°) test in the Fugl-Meyer assessment (FMA) test items, the FMA test may use a method of assigning a point (perfect score: 2 points) to the corresponding test item when the angle of the shoulder formed by the user reaches 90° forward while performing the current scenario. Accordingly, the computing apparatus 101 may continuously recognize the body part of the user in the process of assessing the upper extremity exercise ability of the user using the scenario item. The computing apparatus 101 may identify the posture of the user using a sensor (Kinect, etc.) that may recognize an external full-body joint and may determine the posture score on the upper extremity exercise ability of the user based on the identified posture.

### 2) Second Type

In operation 202, the computing apparatus 101 may perform operation 212 of adjusting the position of an object as a method of testing the exercise function of the hand or wrist of the user. The computing apparatus 101 may generate a virtual image panel having a palm shape or an object similar to the virtual image panel, move the image panel or the object to the position and direction deployable in the virtual space, and then recognize that the user touches the image panel or the object by moving the palm of the user to the corresponding position and direction. For example, the object similar to the virtual image panel may be a covered book, a closed door, etc. The computing apparatus 101 may then induce the sequential movement of the user by moving the virtual image panel again and may identify the exercise function of the wrist of the user through the sequential movement.

### 3) Third Type

In operation 203, the computing apparatus 101 may generate a touchable object by the finger or hand of the user, perform operation 213 of adjusting the position of the touchable object, and analyze the upper extremity exercise ability of the user as the user moves the finger or hand and touches the touchable object. For example, the touchable object may be a butterfly, bee, firefly, etc.

### 4) Fourth Type

In operation 204, the computing apparatus 101 may assess an ability to pick up and lift a certain object. For example, this ability may be spherical grasp. The computing apparatus 101 is an adjustment apparatus and may display a moving position of a controller and/or smartphone in operation 214. That is, the computing apparatus 101 may suggest a goal of picking up and moving a controller used in conjunction with the VR/AR-based visual device or a cell phone generally owned by an individual and may assess a score when the user performs the goal.

In operation 205, the computing apparatus 101 may assess the upper extremity exercise ability of the user using the above-described four types of analysis methods and may simultaneously perform the rehabilitation therapy and assessment. For example, the Reference to determine the upper extremity exercise ability of the user by the score may be performed by selecting some of the 33 FMA items.

Some selected FMA items are as follows. Here, an example of selecting 12 assessment items out of 33 assessment items and performing the 12 assessment items in the rehabilitation content is described.
1) Shoulder Retraction
2) Shoulder Elevation
3) Shoulder Abduction (90°)
4) Shoulder External Rotation
5) Elbow Flexion
6) Wrist-Flexion and/or Extension
7) Elbow Flexion (flexor synergy)
8) Shoulder Flexion to 90°)
9) Spherical Grasp
10) Finger to Nose
11) Wrist Stability
12) Forearm Pronation (extensor synergy)

Items 1) to 5) are flexor synergy and may be checked simultaneously.

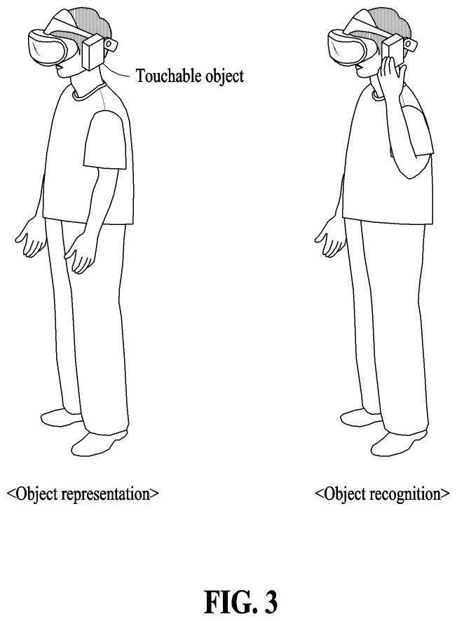

FIG. 3 is a diagram illustrating a method of providing a touchable object through the upper extremity exercise of the user using rehabilitation content, according to an embodiment.

Referring to FIG. 3, a computing apparatus may determine rehabilitation content to assess the upper extremity exercise ability of the user in a pre-set rehabilitation scenario. The computing apparatus may determine a virtual scenario item to interact with the user through the rehabilitation content. When the virtual scenario item is an object, the computing apparatus may set a touchable area by the hand of the user based on body information of the user.

The computing apparatus may set the touchable area by the hand of the user so that the user performs the shoulder retraction according to the purpose of assessing the upper extremity exercise ability of the user. Shoulder retraction is flexor synergy, and the computing apparatus may induce the user to touch a touchable object after moving the touchable object toward the ear in the virtual space in response to the shoulder retraction.

Referring to the right diagram in FIG. 3, the computing apparatus may set the position where the hand of the user is movable based on the body information of the user. For example, the computing apparatus may represent the touchable object at a position where the user brings the hand to the ear.

Referring to the left diagram in FIG. 3, the computing apparatus may recognize the gesture of touching an object with the finger or hand of the user according to the movement of the user in the actual space by interacting with the object displayed in the virtual space. For example, the computing apparatus may recognize whether the object is touched at a position where the user brings the hand to the ear.

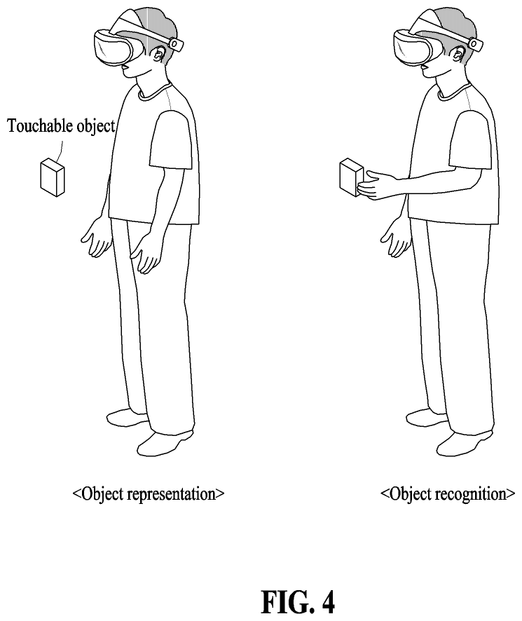

FIG. 4 is a diagram illustrating a method of providing a touchable object through the upper extremity exercise of the user using rehabilitation content, according to another embodiment.

Referring to FIG. 4, when a scenario item is an object, a computing apparatus may set a touchable area by the hand of the user based on body information of the user. The computing apparatus may set the touchable area by the hand of the user so that the user performs the elbow flexion according to the purpose of assessing the upper extremity exercise ability of the user.

Elbow flexion is flexor synergy, and the computing apparatus may recognize the touch on a touchable object of the user in the virtual space after displaying the touchable object at a position as low as the upper arm length from the shoulder height of the user and as long as the forearm length of the user in the virtual space in response to the elbow flexion.

Referring to the right diagram in FIG. 4, the computing apparatus may set a position where the hand of the user is movable based on the body information of the user. For example, the computing apparatus may display the touchable object at a position where the user bends the elbow 90° in the virtual space.

Referring to the left diagram in FIG. 4, the computing apparatus may recognize the gesture of touching an object with the finger or hand of the user according to the movement of the user in the actual space by interacting with the object displayed in the virtual space. For example, the computing apparatus may recognize the touch on the object displayed at a position where the user bends the elbow 90°.

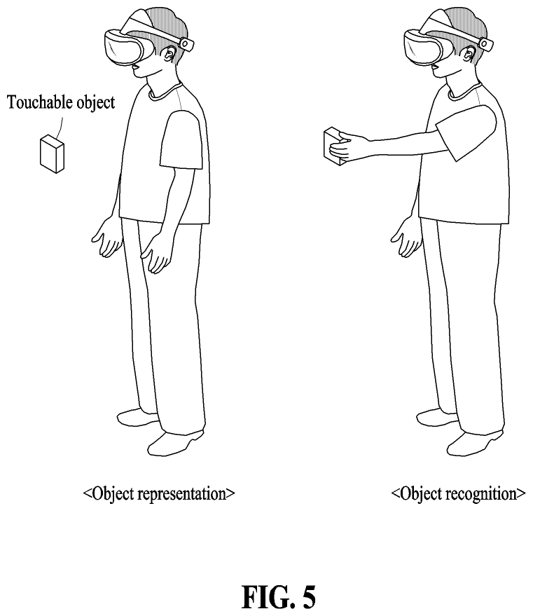

FIG. 5 is a diagram illustrating a method of providing a touchable object through the upper extremity exercise of the user using rehabilitation content, according to another embodiment.

Referring to FIG. 5, when a scenario item is an object, a computing apparatus may set a touchable area by the hand of the user based on body information of the user. The computing apparatus may set the touchable area by the hand of the user so that the user performs the shoulder flexion to 90° according to the purpose of assessing the upper extremity exercise ability of the user.

Shoulder flexion to 90° may be a method of recognizing the touch on a touchable object of the user in the virtual space after displaying the touchable object at a distance at which the angle of the shoulder formed by a patient is 90° and as long as the arm length of the patient in the virtual space.

Referring to the right diagram in FIG. 5, the computing apparatus may set the position where the hand of the user is movable based on the body information of the user. For example, the computing apparatus may display the touchable object at a position where the user extends the arm forward by 90° in the virtual space.

Referring to the left diagram in FIG. 5, the computing apparatus may recognize the gesture of touching an object with the finger or hand of the user according to the movement of the user in the actual space by interacting with the object displayed in the virtual space. For example, the computing apparatus may recognize the touch on the object displayed at a position where the user extends the arm forward by 90°.

Additionally, the computing apparatus may display and recognize the touch on the object according to various purposes in addition to the shoulder retraction, elbow flexion, and shoulder flexion to 90°.

Spherical grasp may be a method of assessing the upper extremity exercise ability of the user by making the user pick up and lift a controller and/or smartphone.

Finger to nose may be a method of assessing the upper extremity exercise ability of the user by recognizing the touch on a touchable object by moving the touchable object forward, moving the touchable object of which the touch is recognized to the nose again, and making the user move the hand to the nose again.

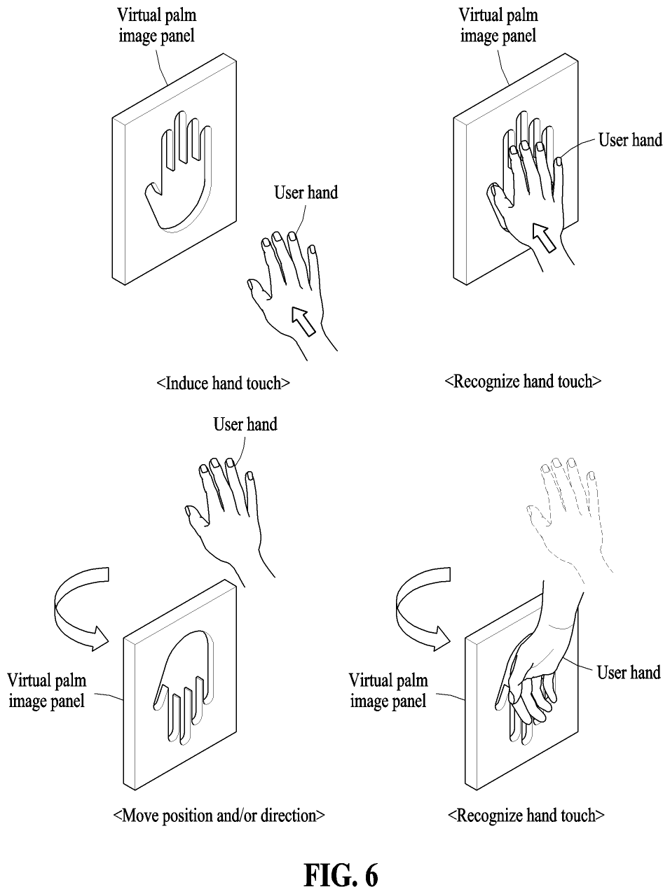

FIG. 6 is a diagram illustrating a method of inducing and recognizing the hand touch of the user using a virtual image panel, according to an embodiment.

Referring to FIG. 6, when a scenario item is an image panel, a computing apparatus may adjust the position and direction of the image panel to be displayed in the virtual space based on body information of the user and spatial information (or an actual space) in which the user is positioned. The computing apparatus may provide the image panel of which the position and direction are adjusted to be displayed in the virtual space to the user through the VR/AR-based visual device.

The computing apparatus may set the image panel in the virtual space so that the user performs the wrist-flexion and/or wrist-extension according to the purpose of assessing the upper extremity exercise ability of the user.

Wrist-flexion and/or wrist-extension is an exercise for moving the wrist of the user up and down, and may be a method of recognizing the result of the user touching the image panel by rotating the palm of the user downward after generating the image panel at a position as long as the arm length of the user so that the palm of the user faces upward, and when the user touches the image panel with the palm, rotating the position and/or direction of the image panel so that the palm of the user faces downward.

Referring to the top right diagram in FIG. 6, the computing apparatus may display the image panel having the palm shape in the virtual space and induce the user to touch the image panel with the palm.

Referring to the top left diagram in FIG. 6, the computing apparatus may recognize whether the user touches the image panel having the palm shape with the palm in the virtual space.

Referring to the bottom right diagram in FIG. 6, the computing apparatus may move the position and/or direction of the image panel having the palm shape in the virtual space.

Referring to the bottom left diagram in FIG. 6, the computing apparatus may re-recognize whether the user touches the image panel with the palm in the virtual space where the position and/or direction of the image panel is moved.

Additionally, the computing apparatus may display and recognize the touch on the object according to various purposes in addition to the wrist-flexion and/or wrist-extension.

Wrist stability may be a method of assessing the upper extremity exercise ability of the user by generating a virtual image panel so that the palm of the user faces upward and making the user maintain the touch when the user touches the virtual image panel with the palm.

Forearm pronation (extensor synergy) may rotate the virtual image panel at a distance as long as the arm length of the user so that the palm of the user faces downward. In addition, when the user touches the virtual image panel with the palm, the forearm pronation may be a method of assessing the upper extremity exercise ability of the user by rotating the virtual image panel so that the palm of the user faces upward and verifying that the user touches the virtual image panel again with the palm.

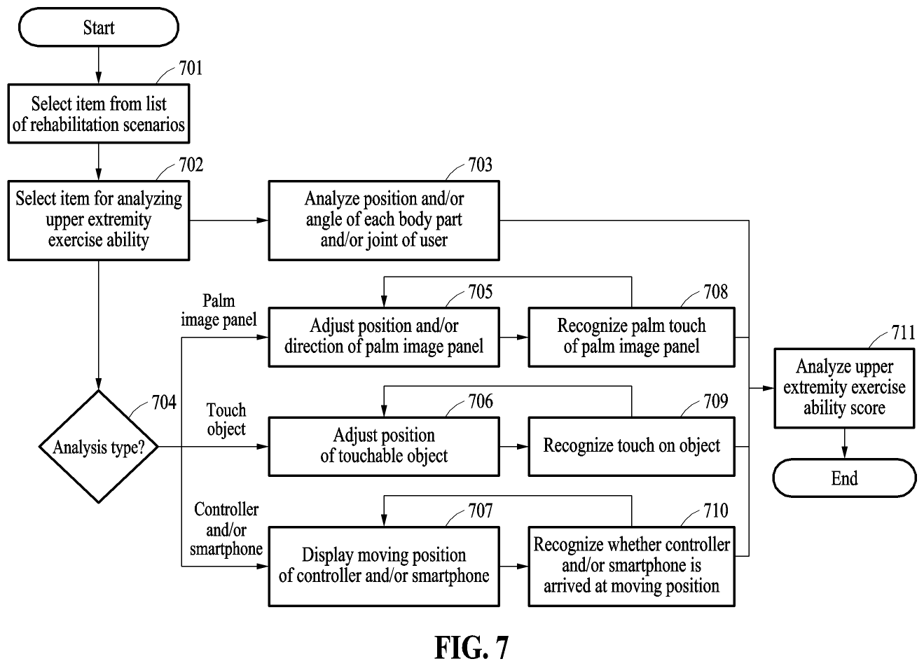

FIG. 7 is a flowchart illustrating a method of assessing an upper extremity, according to an embodiment.

In operation 701, a computing apparatus may select at least one piece of rehabilitation content to be applied to the user from a list of rehabilitation scenarios that may be implemented in various ways. The rehabilitation content may be digital information provided to the user to assess the upper extremity exercise ability of the user.

In operation 702, the computing apparatus may select a scenario item to assess the upper extremity exercise ability of the user through the rehabilitation content selected from operation 701. That is, the computing apparatus may select, as an item existing in the rehabilitation content, a scenario item provided through the VR/AR-based visual device the user wears. The scenario item may include at least one of an image panel represented in the virtual space, a touchable object in the virtual space, and an adjustment apparatus interoperating with the VR/AR-based visual device.

In operation 703, when the scenario item of the rehabilitation content is selected from operation 702, the computing apparatus may analyze the position and angle of each body part and joint of the user. Here, the computing apparatus may interoperate with a VR wearable device attached to the body of the user for interaction in the virtual space in addition to stimulating vision and hearing through the VR/AR-based visual device. The VR wearable device attached to the body of the user may transmit a signal according to the movement of the body part of the user to the computing apparatus. The computing apparatus may analyze the position and angle of each body part and joint of the user by analyzing the signal transmitted from the VR wearable device.

In addition, the VR wearable device may generally recognize the position and direction of the head and eyes of the user using an HMD and recognize the hand part of the user through a dedicated controller or hand recognition. The present disclosure may additionally recognize each joint of the user when a wearable device (e.g., a Vive tracker, etc.) is used together with the VR wearable device. In addition, the present disclosure may recognize the full-body joint when an external recognition device such as Kinect and the like is used together with the VR wearable device.

In operation 704, the computing apparatus may determine a type of the scenario item selected from operation 702.

In operation 705, when the scenario item is an image panel, the computing apparatus may adjust the position and direction of the image panel to be displayed in the virtual space based on body information of the user and spatial information (or an actual space) where the user is positioned that are analyzed in operation 703.

In operation 708, the computing apparatus may provide the image panel of which the position and direction are adjusted to be displayed in the virtual space to the user through the VR/AR-based visual device. The computing apparatus may recognize the gesture of touching the image panel by the palm of the user according to the movement of the user in the actual space by interacting with the image panel displayed in the virtual space. Here, a threshold area may be set in the image panel to assess the upper extremity exercise ability of the user, and the threshold area may be a boundary to distinguish between exercise loads according to a response state of the user in response to the degree of awareness of physical activity. Here, the response state may indicate whether the user touches an image pattern with the palm, and the exercise load may indicate a limitation in which a current state of the palm that touches the image pattern may be maintained.

For example, the threshold area may be a boundary between each state that may be classified into a normal state, an immature state, an abnormal state, etc., according to the position and angle of the palm that touches on the image pattern. The threshold area may be used to assess the upper extremity exercise ability of the user.

The image panel having the palm shape and object may be distinguished from one another by their functionality. The image panel may be represented as a palm shape panel, a three-dimensional (3D) palm model, etc., when the image panel is implemented as content for an actual user. The object may be represented in the form of a butterfly, bee, firefly, etc.

Here, the FMA may assign three levels of points for each motion and may assign 0 points for failure to perform motion, 1 point for partially performing motion, and 2 points for completely performing motion. The computing apparatus may use the sum of each assigned point as a score for the FMA test.

The partially performing motion may indicate that the user does not follow the final motion but performs up to a part of the intermediate path (e.g., up to 50% of the path).

In operation 706, when the scenario item is an object, the computing apparatus may set a touchable area by the hand of the user based on the body information of the user analyzed in operation 703. The touchable area by the hand of the user is a touchable area by the finger of the user, and may be an area requiring fine adjustment, such as the head, eyes, nose, mouth, shoulders, etc.

In operation 709, the computing apparatus may provide the object displayable on the touchable area to the user through the VR/AR-based visual device. The computing apparatus may recognize the gesture of touching the object by the finger or hand of the user according to the movement of the user in the actual space by interacting with the object displayed in the virtual space. Here, the computing apparatus may measure the first time the object is displayed through the VR/AR-based visual device and the second time the object is touched by the finger of the user. In the present disclosure, the reason for measuring the time may be to determine the response rate of the user on the object displayed in the virtual space. The computing apparatus may then be used to assess the upper extremity exercise ability of the user by measuring the difference between the first time and the second time. Additionally, the computing apparatus may calculate the time difference and assign a partial point when the user succeeds in performing a touch operation while exceeding a predetermined time threshold. In addition, when the user fails to fully perform a touch operation while exceeding a predetermined time threshold, the computing apparatus may determine this as a failure.

In operation 707, the computing apparatus may display a moving path of the adjustment apparatus when the scenario item is the adjustment apparatus. The computing apparatus may visually represent the path of movement, that is, the moving path of the adjustment apparatus, based on the position of the user in a user interface through the VR/AR-based visual device.

In operation 710, the computing apparatus may recognize the starting position and the ending position of the adjustment apparatus according to the posture of the user based on the moving path of the adjustment apparatus. The computing apparatus may recognize the movement of the user as to whether the adjustment apparatus held by the hand of the user is moved according to the starting position and the ending position. The computing apparatus may determine the degree of path deviation and direction of path deviation between the starting position and the ending position in response to the moving path visually displayed in the virtual space. Here, the degree of path deviation may be a degree deviating from a range or a line from linearity or a curve displayed as the moving path. The direction of path deviation may be a direction of the left side and right side or upper side and lower side from linearity or a curve displayed as the moving path. The degree of path deviation and direction of path deviation may be used to assess the upper extremity exercise ability of the user.

For example, in the present disclosure, a case in which the intermediate path is not checked may occur in the moving path of the adjustment apparatus, and in this case, the computing apparatus may determine in which the desired motion is satisfied to be a success when starting motion and final motion are satisfied as the desired motion.

In another example, in the case of certain motion (e.g., the wrist-flexion and/or extension), the computing apparatus may determine whether the certain motion is successful by additionally checking the intermediate path in addition to the starting motion and final motion.

In operation 711, the computing apparatus may derive a score on the upper extremity exercise ability of the user by analyzing each result recognized in operations 708 to 710. The computing apparatus may assess the upper extremity exercise ability of the user in response to the derived score.

The method according to embodiments may be written in a computer-executable program and may be implemented as various recording media such as magnetic storage media, optical reading media, or digital storage media.

Various techniques described herein may be implemented in digital electronic circuitry, computer hardware, firmware, software, or combinations thereof. The implementations may be achieved as a computer program product, i.e., a computer program tangibly embodied in an information carrier, e.g., in a machine-readable storage device (for example, a computer-readable medium) or in a propagated signal, for processing by, or to control an operation of, a data processing apparatus, e.g., a programmable processor, a computer, or multiple computers. A computer program, such as the computer program(s) described above, may be written in any form of a programming language, including compiled or interpreted languages, and may be deployed in any form, including as a stand-alone program or as a module, a component, a subroutine, or other units suitable for use in a computing environment. A computer program may be deployed to be processed on one computer or multiple computers at one site or distributed across multiple sites and interconnected by a communication network.

Processors suitable for processing of a computer program include, by way of example, both general and special purpose microprocessors, and any one or more processors of any kind of digital computer. Generally, a processor will receive instructions and data from a read-only memory or a random-access memory, or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Examples of information carriers suitable for embodying computer program instructions and data include semiconductor memory devices, e.g., magnetic media such as hard disks, floppy disks, and magnetic tape, optical media such as compact disk read-only memory (CD-ROM) or digital video disks (DVDs), magneto-optical media such as floptical disks, read-only memory (ROM), random-access memory (RAM), flash memory, erasable programmable ROM (EPROM), or electrically erasable programmable ROM (EEPROM). The processor and the memory may be supplemented by, or incorporated in special purpose logic circuitry.

In addition, non-transitory computer-readable media may be any available media that may be accessed by a computer and may include both computer storage media and transmission media.

Although the present specification includes details of a plurality of specific embodiments, the details should not be construed as limiting any invention or a scope that can be claimed, but rather should be construed as being descriptions of features that may be peculiar to specific embodiments of specific inventions. Specific features described in the present specification in the context of individual embodiments may be combined and implemented in a single embodiment. On the contrary, various features described in the context of a single embodiment may be implemented in a plurality of embodiments individually or in any appropriate sub-combination. Furthermore, although features may operate in a specific combination and may be initially depicted as being claimed, one or more features of a claimed combination may be excluded from the combination in some cases, and the claimed combination may be changed into a sub-combination or a modification of the sub-combination.

Likewise, although operations are depicted in a specific order in the drawings, it should not be understood that the operations must be performed in the depicted specific order or sequential order or all the shown operations must be performed in order to obtain a preferred result. In specific cases, multitasking and parallel processing may be advantageous. In addition, it should not be understood that the separation of various device components of the aforementioned embodiments is required for all the embodiments, and it should be understood that the aforementioned program components and apparatuses may be integrated into a single software product or packaged into multiple software products.

The embodiments disclosed in the present specification and the drawings are intended merely to present specific examples in order to aid in understanding of the disclosure, but are not intended to limit the scope of the disclosure. It will be apparent to those skilled in the art that various modifications based on the technical spirit of the disclosure, as well as the disclosed embodiments, can be made.

What is claimed is:

1. A method of assessing an upper extremity, the method comprising:
determining rehabilitation content to assess an upper extremity exercise ability of a user in a pre-set rehabilitation scenario;
determining a virtual scenario item to interact with the user through the determined rehabilitation content;
providing the virtual scenario item through a virtual reality (VR)/augmented reality (AR)-based visual device the user wears by adjusting a virtual position of the virtual scenario item based on body information of the user;
recognizing movement of the user according to the virtual scenario item provided through the VR/AR-based visual device; and
assessing the upper extremity exercise ability of the user by analyzing the movement of the user.

2. The method of claim 1, wherein the determining of the virtual scenario item comprises determining one virtual scenario item among a virtual object touchable by the user, a virtual image panel, and an adjustment apparatus wearable by the user to assess the upper extremity exercise ability of the user according to a body part of the user.

3. The method of claim 1, wherein the providing of the virtual scenario item comprises:
when the virtual scenario item is a virtual image panel, adjusting a position and a direction of the virtual image panel based on the body information of the user and spatial information where the user is positioned; and
providing the virtual image panel of which the position and the direction are adjusted to the user through the VR/AR-based visual device.

4. The method of claim 1, wherein the providing of the virtual scenario item comprises:
when the virtual scenario item is a virtual object, setting a touchable area by a hand of the user based on the body information of the user; and
providing the virtual object displayable on the touchable area to the user through the VR/AR-based visual device.

5. The method of claim 1, wherein the providing of the virtual scenario item comprises:
when the virtual scenario item is an adjustment apparatus, setting a starting position and an ending position of the adjustment apparatus based on a posture of the user; and
providing the starting position and the ending position of the adjustment apparatus to the user through the VR/AR-based visual device to assess a moving path of the adjustment apparatus held by a hand of the user.

6. The method of claim 3, wherein the recognizing of the movement of the user comprises recognizing movement of the user as to whether the virtual image panel is touched by a palm of the user in response to the position and the direction of the virtual image panel that is visualized through the VR/AR-based visual device.

7. The method of claim 4, wherein the recognizing of the movement of the user comprises recognizing movement of the user as to whether the virtual object is touched by a finger or hand of the user in response to the virtual object that is visualized through the VR/AR-based visual device.

8. The method of claim 5, wherein the recognizing of the movement of the user comprises recognizing movement of the user as to whether the adjustment apparatus held by the hand of the user is moved according to the starting position and the ending position of the adjustment apparatus.

9. The method of claim 1, wherein the assessing of the upper extremity exercise ability of the user comprises:
deriving a score on the upper extremity exercise ability of the user by analyzing movement of the user corresponding to the rehabilitation content based on the pre-set rehabilitation scenario; and
assessing the upper extremity exercise ability of the user in response to the derived score.

10. A computing apparatus for performing a method of assessing an upper extremity, the computing apparatus comprising:
a processor,
wherein the processor is configured to:
determine rehabilitation content to assess an upper extremity exercise ability of a user in a pre-set rehabilitation scenario;
determine a virtual scenario item to interact with the user through the determined rehabilitation content;
provide the virtual scenario item through a virtual reality (VR)/augmented reality (AR)-based visual device the user wears by adjusting a virtual position of the virtual scenario item based on body information of the user;
recognize movement of the user according to the virtual scenario item provided through the VR/AR-based visual device; and
assess the upper extremity exercise ability of the user by analyzing the movement of the user.

11. The computing apparatus of claim 10, wherein the processor is configured to determine one virtual scenario item among a virtual object touchable by the user, a virtual image panel, and an adjustment apparatus wearable by the user to assess the upper extremity exercise ability of the user according to a body part of the user.

12. The computing apparatus of claim 10, wherein the processor is configured to:
when the virtual scenario item is a virtual image panel, adjust a position and a direction of the virtual image panel based on the body information of the user and spatial information where the user is positioned; and
provide the virtual image panel of which the position and the direction are adjusted to the user through the VR/AR-based visual device.

13. The computing apparatus of claim 10, wherein the processor is configured to:
when the virtual scenario item is a virtual object, set a touchable area by a hand of the user based on the body information of the user; and
provide the virtual object displayable on the touchable area to the user through the VR/AR-based visual device.

14. The computing apparatus of claim 10, wherein the processor is configured to:
when the virtual scenario item is an adjustment apparatus, set a starting position and an ending position of the adjustment apparatus based on a posture of the user; and
provide the starting position and the ending position of the adjustment apparatus to the user through the VR/AR-based visual device to assess a moving path of the adjustment apparatus held by a hand of the user.

15. The computing apparatus of claim 12, wherein the processor is configured to recognize movement of the user as to whether the virtual image panel is touched by a palm of the user in response to the position and the direction of the virtual image panel that is visualized through the VR/AR-based visual device.

16. The computing apparatus of claim 13, wherein the processor is configured to recognize movement of the user as to whether the virtual object is touched by a finger or hand of the user in response to the virtual object that is visualized through the VR/AR-based visual device.

17. The computing apparatus of claim 14, wherein the processor is configured to recognize movement of the user as to whether the adjustment apparatus held by the hand of the user is moved according to the starting position and the ending position of the adjustment apparatus.

18. The computing apparatus of claim 10, wherein the processor is configured to:
derive a score on the upper extremity exercise ability of the user by analyzing movement of the user corresponding to the rehabilitation content based on the pre-set rehabilitation scenario; and
assess the upper extremity exercise ability of the user in response to the derived score.

19. A system for assessing an upper extremity, the system comprising:
a virtual reality (VR)/augmented reality (AR)-based visual device configured to play immersive rehabilitation content in a space where a user is positioned; and
a computing apparatus configured to assess an upper extremity exercise ability of the user by recognizing movement of the user through the VR/AR-based visual device,
wherein the computing apparatus is configured to:
provide rehabilitation content through the VR/AR-based visual device the user wears based on body information of the user; and
assess the upper extremity exercise ability of the user by analyzing movement of the user according to the rehabilitation content provided through the VR/AR-based visual device.
